(19) United States
(12) Patent Application Publication
(10) Pub. No.: US 2005/0214365 A1
(43) Pub. Date: Sep. 29, 2005

(54) [Instant dissolving tablet composition for loratidine and desloratidine]

(75) Inventors: Abdul Razzaq Yousef, Ras Al-Khaimah (AE); Quamar Zaman, Ras Al-Khaimah (AE)

Correspondence Address: SARFARAZ K. NIAZI, 20 RIVERSIDE DRIVE, DEERFIELD, IL, 60015, US

(73) Assignee: GULF PHARMACEUTICAL INDUSTRIES, Ras Al-Khaimah (AE)

(21) Appl. No.: 10/708,773

(22) Filed: Mar. 24, 2004

(51) Int. Cl. A61K 9/20; A61K 9/46

(52) U.S. Cl. 424/464; 514/290

(57) ABSTRACT

Disclosed here is a tablet formulation of loratidine and desloratidine, non-sedating antihistaminic agents, that allows fast dissolution of tablets in the mouth allowing administration of these drugs without the aid of water. The formulation has pleasing taste and texture.

### BACKGROUND OF INVENTION

Loratidine is disclosed in U.S. Pat. Nos. 4,282,233, 4,659,716, and 4,863,931 as a non-sedating antihistamine useful for treating allergic reactions. Loratidine (ethyl-[4-(8-chloro-6,11-dihydro-5H-benzo[5,6]cyclohepta [1,2-b]pyridin-11-ylidene)-1-piperidinecarboxylate]) is an antihistamine that is available commercially as syrup or in the form of tablets. (See also Claritin brand of Loratadine. Product Information Sheet, dated January 1999.) It is available in both as standard dosage and in sustained release dosage form as well as in fast dissolving oral tablets. Following oral administration, loratadine is rapidly metabolized to descarboethoxyloratadine or desloratadine (also disclosed in U.S. Pat. No. 6,100,274), a pharmacologically active metabolite. Both loratidine and desloratidine are nonsedating antihistaminic agents used widely for a variety of allergic disorders such as for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal and perennial allergic rhinitis, including nasal congestion, in patients in need of such treating and/or preventing. Both drugs are also useful for the treatment of chronic idiopathic urticaria. The use of loratidine and desloratidine is frequently made on as needed basis and in many instances on a regular daily basis. Whereas the preferred drug delivery system for administering loratidine and desloratidine is an oral tablet, it is often difficult to swallow tablets by a large number of patients, particularly the elderly and the children; the tablet dosage forms further require the use of a suitable liquid to swallow, which may not be readily available. To obviate both of these drawbacks in the use of the preferred dosage form for oral use, there is a need to formulate a tablet that will disintegrate quickly in the mouth within seconds after placement on the tongue, allowing its contents to be subsequently swallowed without water, making the use of water in swallowing the tablet an optional feature. Whereas attempts have been made to formulate such products, there remains a need to formulate a product that will be good tasting, having a good feel in the mouth and readily and consistently dissolved to provide a consistent response in the body. There is a need to produce pharmaceutical compositions suitable for oral administration to mammals containing loratidine or desloratidine having constant chemical and physical properties in accordance with exacting health registration requirements of the U.S. and international health registration authorities, e.g., the FDA Good Manufacturing Practices Requirements and the International Conference on Harmonization (ICH) Guidelines. Such products are not currently available to consumers

### SUMMARY OF INVENTION

Dosage form designs that are designed to deliver drugs through mouth, buccal, sublingual, chewable, etc., are faced with the significant problem of masking the taste of drugs. Active drugs have, by their nature, active chemical functional groups, which then interact with the taste buds and create strong sensation of taste. As a result drugs are often administered in tablets which are coated using polymers of sugar to mask the taste and smell of drugs. As a result, few drugs have been administered by the route of oral cavity despite many advantages provided by this route of administration. The advantage includes faster absorption as a result of high blood flow in the oral cavity and the bypassing of absorbed active drug moieties from the first pass effect in the liver, of the portion of drug that gets directly absorbed from the oral cavity into blood. However, in almost all instances when drugs are administered in the oral cavity, portions of drugs are swallowed and pass through the gastrointestinal tract; this effect is reduced where the drug is applied as a device to the oral cavity. Regardless, the variation in the portion of drug being absorbed directly to the circulation from the oral cavity and the portions entering the gastrointestinal tract create inconsistencies in the drug response, if the drug is significantly metabolized in the liver, or is subject to bioavailability variations when administered orally or when the drug is decomposed in the gastrointestinal tract. There is also a significant problem of drugs irritating oral mucosa when they are administered through this route of administration. Further, many dosage forms intended for chewing or for keeping in mouth for any length of time often produce an unpleasant feeling, related to taste and texture; this results in lack of patient compliance. A good dosage form that intends to deliver the drug to mouth should therefore posses many characteristics including but not limited to pleasant taste, taste masking, non-gritty feeling, fast dissolving and not leaving any taste behind. In addition, the dosage form must deliver the drug consistently. It is often impossible to combine all of these characteristics for drugs based on any fixed formulae or lessons learned from such similar formulations for other drugs. In this invention, we have experimented and perfect a surprisingly pleasant taste, non-gritty feel, fast dissolving tablet that consistently delivers the exact amount of required dose of loratidine and desloratidine. The invention is based on the choice of a novel disintegrant, PHARMABURST a proprietary formulation based on mannitol. A choice of various flavoring agents, lubricants and sweetening agents was necessary to obtain the desired characteristics. We have achieved these results in a surprisingly simple formula that is also cost effective. The subject composition can be administered to all patients, from children to elderly and does not require use of water to swallow the contents.

### DETAILED DESCRIPTION

A large number of formulations were prepared varying the amounts of each of the inactive ingredients to establish an optimal formulation. It was found that there was no linearity in the features of the formulation obtained that could be projected based on prior art, the art common to formulation scientists and other academic and commercial sources. It was found surprisingly that more than one lubricant in specific quantity is needed to impart the tabletting characteristics to the powder mixture. The flavors used and their specific combinations could not be predicted based on prior art, nor from the trial and error basis; the surprising results in improving the taste of the formulation were obtained by adjusting these flavor quantities. Additionally, the use of a proprietary disintegrant, PHARMABURST, allowed imparting all characteristics necessary to achieve the quick dissolving nature, an appropriate feeling of flavor and texture.

Given below is a typical manufacturing formula for the instant invention:

Formula for 500,000 tablets

| Ingredient | Quantity (kg) |
|---|---|
| Loratidine micronized | 5.00 |
| PHARMABURST | 84.72 |
| Acesulfame potassium | 1.30 |
| Anise dry flavor | 1.20 |
| Mint dry flavor | 0.15 |
| Talc fine powder | 2.00 |
| Magnesium stearate | 2.00 |
| Silicon dioxide | 1.50 |
| Stearyl fumarate | 2.12 |

Method of Manufacture: The quantity of loratadine or desloratidine micronized is calculated based on 100% assay; actual quantity is calculated based on the assay result. The amount is therefore adjusted accordingly. The precautions in the manufacturing of this product including wearing a fast mask and hand gloves to avoid exposure to lungs and to skin. Throughout the manufacturing process, the temperature should be maintained between 20-25 C and humidity of not more than 30%. Dry PhHARMABURSTn a Lytzen oven for 10 hours at 55 C. Check loss on drying and assure that it is no more than 0.5%. Dry more if necessary until the desired loss on drying is achieved. Sift loratadine micronized, acesulfame potassium and PHARMABURST twice through a stainless sieve of 500 micron in Russell Mixer. Load into drum blender. Mix for 5 minutes. Sift anise dry flavor, mint dry flavor, talc fine powder, magnesium stearate, silicon dioxideand sodium stearyl fumarate through a stainless sieve of 250 micron size in Russell mixer. Load into drum blender. Mix for 5 minutes. Compress the granules using rotary tabletting machine using 8.0 mm, bevel, and concave upper and lower punch. The weight of 10 tablets should be 2.00 g, plus or minus, 3%, weight variation no more than 5% of average weight, thickness of 4, plus or minus 0.3 mm and hardness 4-8 kp, and friability not more than 1%.

1. A pharmaceutical composition for oral administration comprising of an anti-allergic effective amount of loratadine in a pharmaceutically acceptable carrier medium consisting essentially of a proprietary disintegrant, PHARMABURST, an amount of lubricant talc, an amount of lubricant sodium stearyl fumarate, an amount of lubricant magnesium stearate, an amount of lubricant silicon dioxide, an amount of sweetening agent acesulfate potassium, an amount of flavor anise dry flavor, and an amount of flavor mint dry sufficient to provide dissolution of at least about 80% by weight of the pharmaceutical composition in about 45 minutes.

2. The pharmaceutical composition of claim 1 wherein the loratidine:disintegrant ratio is from 1:5 to 1:20 on a weight by weight basis.

3. The pharmaceutical composition of claim 1 wherein loratidine is substituted by desloratidine.

4. The pharmaceutical composition of claim 1 wherein the pharmaceutically acceptable basic salt is a calcium, magnesium or aluminum salt, or mixtures thereof.

5. A pharmaceutical composition of claim 1 which comprises (as weight, %), Loratidine micronized, about 0.5-15; PHARMABURST, 80-90; acesulfame potassium, 1-2; anise dry flavor, 1-2; mint dry flavor, 0.1-0.2; talc fine powder,1-3; magnesium stearate, 1-3; silicon dioxide, 1-2; sodium stearyl fumarate, 2-3.

6. A pharmaceutical composition of claim 1 which comprises loratidine 10.00 mg, PHARMABURST 169.40 mg, acesulfame potassium 2.60 mg, anise dry flavor 2.40 mg, mint dry flavor 0.30 mg, talc fine powder 4.00 mg, magnesium stearate 4.00 mg, silicon dioxide 3.00 mg, sodium stearyl fumarate 4.25 mg.

7. The pharmaceutical composition of claim 1 wherein said composition is adapted for oral administration.
